(19) United States
(12) Patent Application Publication
(10) Pub. No.: US 2016/0188810 A1
(43) Pub. Date: Jun. 30, 2016

(54) METHOD AND DEVICE FOR THE DEFINITIVE ASSIGNMENT OF MEDICAL MEASURING DEVICES

(71) Applicant: seca ag, Reinach BL 1 (CH)

(72) Inventor: Rüdiger LEUNER, Hamburg (DE)

(21) Appl. No.: 14/984,828

(22) Filed: Dec. 30, 2015

(30) Foreign Application Priority Data
Dec. 30, 2014 (DE) 10 2014 019 594.5

(51) Int. Cl. G06F 19/00; G06K 7/10; G06K 7/14; A61B 5/00

(52) U.S. Cl. CPC G06F 19/323; A61B 5/0017; A61B 5/0022; A61B 5/742; G06K 7/10722; G06K 7/10554; G06K 7/1413; A61B 2562/08; A61B 2560/0475

(57) ABSTRACT

A measurement recording device and a method for acquiring patient measurement values and transmitting them to a display and operating unit and/or a central unit. The device including at least one measurement recording sensor for measuring body properties and/or body functions. The measurement recording device can be identified by at least one ID code and cooperates functionally with at least one scanning device for detecting the ID code or ID feature of a patient and/or for detecting the at least one ID code of the measurement recording device, so that an error-reduced assignment or mix-up of the ID code or ID feature of a patient and his measured body property or body function is supported.

### CROSS-REFERENCE TO RELATED APPLICATIONS

The present application claims priority of DE 10 2014 019 594.5, filed Dec. 30, 2014, the priority of this application is hereby claimed and this application is incorporated herein by reference.

### BACKGROUND OF THE INVENTION

The invention pertains to a measurement recording device and to a method for acquiring patient measurement values and transmitting them to a display an operating unit and/or central unit, comprising at least one measurement recording sensor for measuring properties and/or functions of the body.

Both mobile and stationary measuring devices are used to measure body properties and body functions of subjects, e.g., humans or animals. The properties or functions to be measured often consist of the bioimpedance, i.e., the impedance of the body, as well as heart functions, nerve functions, and electrocardiograms. Other measurement variables in this context can be body weight, body compositions, geometric dimensions, fingerprints, body temperatures at various locations, composition of the utilized respiratory gas, respiratory gas volumes, blood pressure values, etc.

To make these measurements, the measuring device must be connected to the subject to be measured, or the measurement variables in question must be measured or acquired in some other suitable fashion; the values are then frequently processed and transmitted elsewhere. Whether a measurement can be made by contact with the patient or without such contact depends on the property, body function, or vital parameter to be measured and also on the measurement method used for the purpose.

The available measurement technology in general and current medical technology in particular make it possible to determine an increasingly large number of vital parameters, body properties, and body functions in increasingly shorter periods of time and to process these values electronically.

To keep examination costs low, it is necessary to have economically optimized processing and evaluation methods for each of the various measurement variables. To achieve this goal, combinations of devices consisting of a measurement recording device and an associated measurement value processing and evaluation device are going out of use; this is true in particular in hospitals and large joint practices, where a very large number of measurement values must be recorded from a large number patients. Instead, the recorded measurement values acquired by a great many measurement recording devices are now usually being sent to a central measurement processing and evaluation device.

This procedure is a consequence of the fact that an increasingly large number of functionalities must be carried out by intelligent sensors without the need for complicated display and operating units, and instead of using individual measuring devices to process the measurement values in decentralized fashion, increasing use is being made of central evaluation, display, and operating units which are appropriately networked with each other. In addition, it must be possible to assign the measurement recording devices, also called “satellites”, definitively to a specific patient.

For the later evaluation of the measurement data, it is necessary for both the patient and the measuring device used to be definitively assigned to a specific data set. For the diagnosis and determination of a suitable treatment to cure the disease of the patient in question, it is necessary for this assignment to be absolutely correct; a database which is incorrect with respect to its content or assignment can have extremely negative consequences.

A situation can exist, for example, in which the same measurement recording device is used in succession for different patients; it is also possible for different measurement recording devices to make successive measurements of the same patient. The performance of the individual measurements often occurs before the attending physician has the opportunity to discuss the treatment with the patient.

In hospitals, large practices, and other treatment facilities with large numbers of patients, i.e., large numbers of persons to be measured, the standard procedure is to provide each patient or each person with an identification device, which is often determined by an armband and which contains an ID code. The ID code can be an ID number in the form of alphanumeric symbols printed on the armband.

Alternatively or in addition to an ID number, an ID code can be in the form of a preferably machine-readable barcode, a color sequence combination, or some other suitable ID code. In addition to the use of a so-called patient armband, it is also possible, alternatively or in addition, to use one of the definitive properties of the body of the patient such as fingerprints or the geometric features and colors of the iris of the eye.

When a patient, who can now be identified by the identification device, is sent to a certain location to be measured, i.e., to have a measurement value recorded, the measurement values are usually assigned by the operators of the measuring devices; or, in the case of a machine-readable ID code, preferably a code which can be scanned optically or by a sensor, or in the case of the definitive identification feature, the measurement values are assigned by a separate measurement recording device appropriate to the purpose.

If the measurement values are assigned by the operators of the measuring devices, errors and incorrect measurement value assignments, reading errors, or mix-ups are a constantly recurring source of error.

If the measurement values are assigned by a separate measurement recording device appropriate to the purpose, this device is realized in the form of, for example, a barcode scanner or a sensor, which records the ID code or the ID feature, which code or feature must be machine-readable. This method is more reliable than the assignment of the measurement values by the operators of the measuring devices, but incorrect assignments cannot be completely excluded here either because of the involvement of human actions. Once an incorrect assignment has been made, it is in practice impossible to detect the assignment error or to discover it by checking.

### SUMMARY OF THE INVENTION

It is therefore the goal of the invention to provide a method and a device for assigning measurement values to the specific patient to be measured, which method and device increase the reliability of the measurement value assignment overall, improve the ability to check the assignment for correctness, and support a low-cost setup and ease of use.

According to the invention, this goal is achieved by the identifiability both of the patient and of the at least one measurement recording device for measuring body properties and body functions by means of at least one ID code, wherein the measurement recording device cooperates functionally with at least one scanning device to detect the ID code or the ID feature of a patient and/or to detect the at least one ID code of the measurement recording device, so that an error-reduced assignment or mix-up of the ID code or ID feature of a patient and his measured body property and/or body function is supported, as well as the linking of the measurement values in question and scanning results as a method for measurement data handling.

The invention understands that the disadvantages of the prior art can be at least reduced by equipping the satellite or its sensors containing the at least one measurement recording device for measuring body properties and body functions with an ID code, which allows its definitive identification, and by functionally or physically combining it with at least one measurement recording device for scanning both the ID code or the ID feature of the patient and the ID code of the at least one measurement recording device for measuring body properties and body functions. Barcode scanners and optical detection devices such as cameras and opto-electronic detection systems, for example, are suitable as scanning devices.

The ID code or the ID feature of the patient and the body properties or functions to be measured in a specific case and/or the ID code of the measurement recording device for measuring body properties and functions can, according to the invention, be measured at the same time or in a timed sequence and jointly transmitted as a data packet to a central control and display unit either immediately or after a certain delay, having first been stored on the satellite and held for later transmission.

As a result of the joint measurement and joint storage or transmission, either simultaneous or staggered, of the patient measurement values, of the ID code or ID feature of the patient, and/or of the ID code of the measurement recording device (configured preferably as a satellite) for measuring physical properties or body functions, an incorrect assignment or mix-up of ID codes or ID features and the associated physical property or body function is prevented.

Another special advantage of the invention consists in the possibility of checking the assignment for correctness. In addition to the assignment of the measurement data to a patient on the basis of the linking of the measurement values in question with the scanning results, the ID result of the measurement recording device which performed the measurements is also added to this data packet. According to the invention, three sets of data are linked: the measurement data themselves, the ID data of the identified patient, and the ID data of the identified measurement recording device. Assignments can be checked for correctness by means of plausibility checks, either of a time-based or location-based nature, or on the basis of the measurement method required for the measurement in question. It is possible to reconstruct definitively which measurement values were acquired from which patient by what measurement recording device.

Through the use of the invention, it is possible to use relatively simple measurement recording devices without hardware-implemented intelligence. When the data are evaluated after the measurement procedure, it is possible to differentiate between valid measurement data and invalid data before or after the they are transmitted. If the data evaluation flags the presence of an error or a discrepancy, a repeat measurement can be initiated immediately.

In the performance of an identification process, the same scanning device can be used both to scan the ID code of the measurement recording device and to scan the ID code or the ID feature of the patient, provided that both of these make a similar type of scanning possible. If, for example, the ID code of the measurement recording device and the ID code or the ID feature of the patient are realized as optically scannable barcodes, then a barcode scanner can be used for the ID procedures.

To support the error-free assignment of the measurement values, the invention provides several work steps, the order of which can, under certain conditions, vary: In the first step, the ID code of the measurement recording device is scanned or acquired. Then, in the second step, simultaneously or staggered, the ID code of the patient is scanned, and his measurement values are recorded. After that, in step 3, the ID code of the measurement recording device is scanned or acquired again.

The results of this process are three data packets, consisting of the ID information for the measurement recording device, the ID information for the patient, and the measurement values in the form of vital parameters and/or various body properties or body functions. Even before they are transmitted to the display and operating unit or to the central unit, the data packets are already linked together within the measurement recording device, which is frequently configured as a satellite, and/or within the scanning device or the at least one sensor.

The invention supports both the simultaneous, i.e., immediate, and the staggered transmission of the measurement variables and/or the scanning results to a display and operating unit or central unit. To allow the staggered transmission of the measurement variables of body properties and body functions or the scanning results in the form of the ID code or ID feature, a suitable data storage unit is provided in the satellite.

The data transfer between the satellite and the display and operating unit or central unit can be based on various wired or wireless methods; it can, for example, be carried out electrically, optically, by radio, by WiFi, over a network, via Bluetooth, etc.

The various features of novelty which characterize the invention are pointed out with particularity in the claims annexed to and forming a part of the disclosure. For a better understanding of the invention, its operating advantages, specific objects attained by its use, reference should be had to the drawings and descriptive matter in which there are illustrated and described preferred embodiments of the invention.

### BRIEF DESCRIPTION OF THE DRAWING

In the drawing:

FIG. 1 shows an example of a scanning device (30) in the form of a compact module for scanning barcodes;

FIG. 2 show an example of a scanning device (30) in the form of a compact camera module;

FIG. 3 shows by way of example the concept of the method according to the invention consisting of a display and operating unit (10) and, set off from that, i.e., physically separated from it, the measurement electronics in the form of a satellite (20); and

FIG. 4 shows by way of example the concept of a satellite (20), configured as a measurement recording device for measuring body properties and body functions and for scanning the ID code or the ID feature, and consisting essentially of a measuring mat (21) comprising at least one sensor (22) for measuring body properties and body functions and a scanning device (30) for detecting the ID code (40) of the measurement recording device and the ID code or ID feature of the patient.

### DETAILED DESCRIPTION OF THE INVENTION

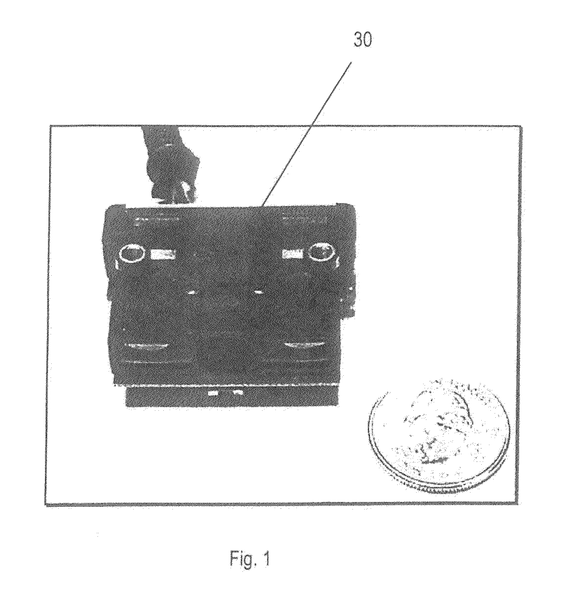

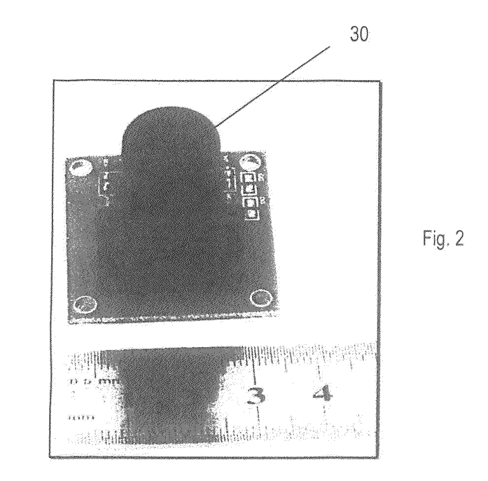

FIGS. 1 and 2 show by way of example possible embodiments of the scanning device (30) for detecting ID codes or ID features in the form of compact modules based on the optical operating principle.

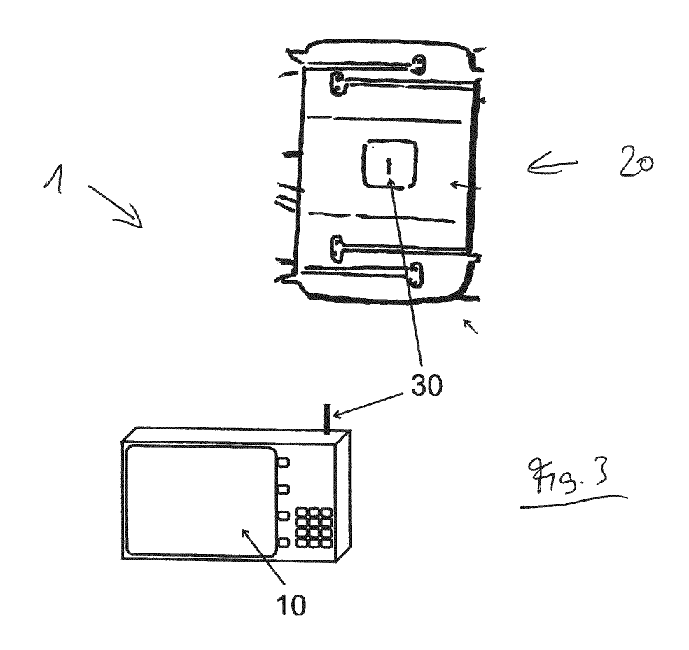

FIG. 3 shows by way of example the concept consisting of a display and operating unit (10) and, set off from that, i.e., physically separated from it, the measurement recording device in the form of a satellite (20). With this concept, the required economically optimized processing and evaluation of the measurement variables in question is achieved. Especially in applications involving a large number of measurement values to be recorded for one or a large number of patients, it is possible in this way, using a large number of satellites (20), to send the recorded measurement values to a usually central measurement processing and evaluation device (10).

This concept takes into account the fact that more and more functionalities must be executed by intelligent sensors without complicated display and operating units and that, instead of decentralized measurement processing by individual measuring devices, increasing use is being made of central evaluation, display, and operating units with appropriate networking.

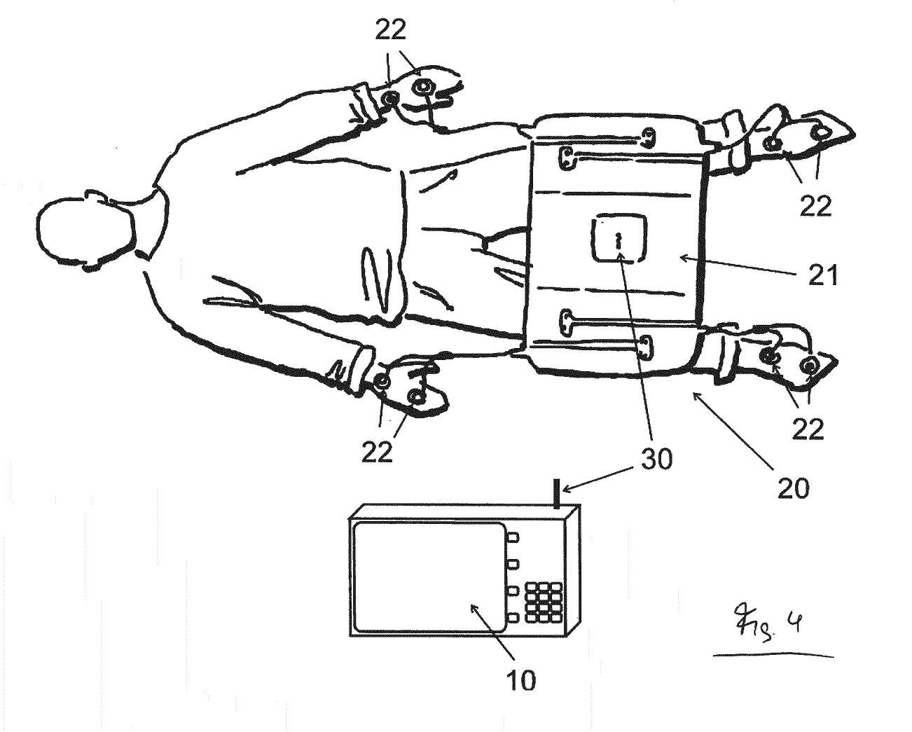

FIG. 4 illustrates by way of example the concept of a satellite (20) according to the invention configured as a measurement recording device for measuring body properties and body functions and for scanning ID codes or ID features, consisting essentially of a measuring mat (21) comprising at least one sensor (22) for measuring body properties and body functions and a scanning device (30) for detecting the ID code or ID feature of the patient and/or of the measurement recording device (20).

In the usual case, one scanning device (30) for detecting the ID code or the ID feature per satellite (20) is sufficient. FIG. 4 shows various positioning possibilities. The scanning device (30) in the example shown here is set up in the area of the measuring mat (21) and/or in the area of at least one sensor (22) and thus is a component of the measurement recording device (20).

If the scanning device (30) must scan both the ID code or ID feature of the patient and the ID code of the measurement recording device (20), the arrangement must be made accordingly in such a way that this is supported. Another possible solution consists in an independent thus separate scanning device (30).

As a measurement recording device, the satellite (20) can be based on various types of devices for measuring body properties and body functions, which can be combined with at least one device for scanning (30) the ID code or the ID feature of the patient and/or the ID code of the measurement value recording device (20).

The measuring mat (21) shown by way of example in FIG. 4 as a basis for the satellite (20) offers handling advantages. Because the measuring mat (21) lies crosswise over the legs of the patient while the measurements are being made, the patient can easily reach the ends of the measuring mat (21), on which the least one scanning device (30) for detecting the ID code or ID feature of the patient is in its preferred position. The ID code can be scanned in from the patient's armband, or an ID feature such as the iris, for example, can be easily detected optically, especially with the active cooperation of the patient.

FIG. 4 also shows, on the basis of various positioning examples, different possibilities, either alternatively or in addition, for arranging the ID code (40) of the measurement recording device (20). Conceivable positions are suitable points on or in the measurement mat (21) and/or on at least one of the sensors (22). If at least one ID code (40) is provided on the sensor (22), it is then possible not only to assign the measurement value to a measurement recording device (20) but also to assign it to an individual sensor (22).

While specific embodiments of the invention have been shown and described in detail to illustrate the inventive principles, it will be understood that the invention may be embodied otherwise without departing from such principles.

We claim:

1. A measurement recording device for acquiring patient measurement values and transmitting them to a display and operating unit and/or a central unit, the measurement recording device comprising at least one measurement recording sensor for measuring properties and/or functions of a body, wherein the measurement recording device is identified by at least one ID code, wherein the measurement recording device cooperates functionally with at least one scanning device for detecting an ID code or ID feature of a patient and/or for detecting the at least one ID code of the measurement recording device, so that an error-reduced assignment or mix-up of the ID code or ID feature of the patient and a measured body property or body function is supported.

2. A measurement recording device according to claim 1, wherein the ID code of the measurement recording device is configured so that the ID code of the measurement recording device and the ID code or ID feature of the patient is scannable by the at least one scanning device.

3. A measurement recording device according to claim 1, wherein the at least one scanning device is based on an optical or opto-electronic operating principle.

4. A measurement recording device according to claim 1, wherein the ID code of the measurement recording device is a barcode.

5. A measurement recording device according to claim 1, wherein the measurement recording device is configured as a satellite, so that an autonomous measurement of body properties and/or body functions and/or the detection of the ID code of the measurement recording device and/or the detection of the ID code or ID feature of the patient is supported.

6. A measurement recording device according to claim 1, wherein the measurement recording device for measuring body properties and/or body functions of a patient and/or the at least one scanning device is integrated into a measuring mat.

7. A measurement recording device according to claim 6, wherein the at least one scanning device is positioned in the measurement mat so that both the ID code of the measurement recording device and the ID code or ID feature of the patient are scanned.

8. A measurement recording device according to claim 1, wherein the at least one scanning device is configured as a module operating on an optical or opto-electronic principle.

9. A measurement recording device according to claim 1, wherein the at least one scanning device is a barcode scanner or a camera.

10. A measurement recording device according to claim 1, further comprising at least one data storage device for temporary or permanent storage of measurement values of the measurement recording device and/or of the at least one scanning device.

11. A measurement recording device according to claim 1, further comprising a wireless transmission device for communication and/or data transmission between the at least one measurement recording device and an evaluation, display, and/or operating unit.

12. A measurement recording device according to claim 11, wherein the wireless transmission device is formed by an infrared, WiFi, or Bluetooth interface.

13. A measuring system for acquiring and evaluating measurement data consisting of body properties, body functions, and/or vital parameters of a patient, comprising at least one measurement recording device for acquiring measurement data according to claim 1; and at least one evaluation, display, and/or operating unit for evaluating and further processing the measurement data.

14. A method for acquiring patient measurement values and transmitting them to a display and operating unit or a central unit, the method comprising the steps of:
(a) measuring body properties and/or body functions of a patient;
(b) detecting an ID code and/or an ID feature of the patient to be measured;
(c) transmitting the measured and detected data to a display and operating unit or a central unit; and
(d) detecting an ID code of a measurement recording device, wherein measured and/or detected data packets are linked into one data packet, so that an error-reduced assignment or mix-up of the ID code or ID feature and the associated body property and/or body function in question is supported.

15. A method for acquiring patient measurement values and transmitting them to a display and operating unit or a central unit according to claim 14, wherein step (d) is executed first and is placed before steps (a)-(c).

16. A method for acquiring patient measurement values and transmitting them to a display and operating unit or a central unit according to claim 14, further comprising carrying out a step (e), which is essentially similar to step (d), so that the detection of the ID code of the measurement recording device is carried out twice.

17. A method for acquiring patient measurement values and transmitting them to a display and operating unit or a central unit according to claim 16, wherein step (e) is carried out after steps (a)-(d).

18. A method for acquiring patient measurement values and transmitting them to a display and operating unit or a central unit according to claim 14, wherein steps (a)-(d) are carried out simultaneously.

19. A method for acquiring patient measurement values and transmitting them to a display and operating unit or a central unit according to claim 16, wherein at least one of steps (a) and (b) is carried out at a time different from that of step (d) and/or step (e), wherein at least one data storage device of the measurement recording device temporarily or permanently stores the measurement values of sensors and/or of at least one scanning device.

20. A method for acquiring patient measurement values and transmitting them to a display and operating unit or a central unit according to claim 14, wherein the order of steps (a) and (b) is reversed.

21. A method for acquiring patient measurement values and transmitting them to a display and operating unit or a central unit according to claim 16, wherein the detection steps (b), (d), and (e) are carried out by optical or opto-electronic scanning, so that a scanning device is used for all detection steps.

22. A method for acquiring patient measurement values and transmitting them to a display and operating unit or a central unit according to claim 16, wherein measurement results from step (a) and detection results from step (b) are linked together.

23. A method for acquiring patient measurement values and transmitting them to a display and operating unit or a central unit according to claim 22, wherein linked results of steps (a) and (b) are linked with the detection results of step (d).

24. A method for acquiring patient measurement values and transmitting them to a display and operating unit or a central unit according to claim 23, wherein an assignment of the results of steps (a), (b), and (d) is checked for correctness through a linkage with detection results of step (e), wherein the ID codes detected in steps (d) and (e) must be identical.

25. A method for acquiring patient measurement values and transmitting them to a display and operating unit or a central unit according to claim 23, wherein the assignment checking is carried out by a time-based or location-based plausibility check.

26. A method for acquiring patient measurement values and transmitting them to a display and operating unit or a central unit according to claim 14, wherein at least one of the detection or measurement steps is carried out simultaneously with step (c).

27. A method for acquiring patient measurement values and transmitting them to a display and operating unit or a central unit according to claim 14, wherein step (c) is realized by a wireless transmission method.
